(19) United States
(12) Patent Application Publication
(10) Pub. No.: US 2023/0293116 A1
(43) Pub. Date: Sep. 21, 2023

(54) VIRTUAL ASSIST DEVICE

(71) Applicant: Hani Esmaeili, Pleasant Hill, CA (US)

(72) Inventor: Hani Esmaeili, Pleasant Hill, CA (US)

(21) Appl. No.: 18/186,140

(22) Filed: Mar. 17, 2023

Related U.S. Application Data
(60) Provisional application No. 63/269,608, filed on Mar. 18, 2022.

(51) Int. Cl. A61B 5/00; A61B 5/308; A61B 5/31; A61B 5/313

(52) U.S. Cl. CPC A61B 5/7267; A61B 5/308; A61B 5/31; A61B 5/313; A61B 5/7405; A61B 5/7455

(57) ABSTRACT

According to an aspect of an embodiment, a virtual assist device may comprise an agent, an input component, and a communication unit. The agent may be configured to receive an incoming notification and request an input in response to the incoming notification. The input component may be an electroencephalogram (EEG) input component that may be configured to receive the input comprising EEG data and send the input to the agent. The agent may be configured to determine a command based on the input. The communication unit may be configured to cause an action to be performed based on the command.

### RELATED APPLICATION

This application claims the benefit of U.S. Provisional Application No. 63/269,608, filed Mar. 18, 2022, the disclosure of which is incorporated herein by reference in its entirety.

### FIELD

The embodiments discussed in the present disclosure relate to a virtual assist device, including a wearable assist device.

### BACKGROUND

Unless otherwise indicated herein, the materials described herein are not prior art to the claims in the present application and are not admitted to be prior art by inclusion in this section.

Communication technology has increased the number of notifications that users receive on devices. Emails, text messages, calendar notifications, group text notifications, phone calls from known and unknown numbers, and the like provide constant interruptions. In some cases, a personal assistant may monitor the notifications and respond accordingly. However, hiring a personal assistant is impractical for many people.

The subject matter claimed in the present disclosure is not limited to embodiments that solve any disadvantages or that operate only in environments such as those described above. Rather, this background is only provided to illustrate one example technology area where some embodiments described in the present disclosure may be practiced.

### SUMMARY

In some embodiments, a virtual assistant device may comprise an agent, an input component, and a communication unit. The agent may be configured to receive an incoming notification and request an input in response to the incoming notification. The input component may be an electroencephalogram (EEG) input component that may be configured to receive the input comprising EEG data and send the input to the agent. The agent may be configured to determine a command based on the input. The communication unit may be configured to cause an action to be performed based on the command.

In some embodiments, a computer-readable storage medium may include computer-executable instructions that, when executed by one or more processors, may cause an agent to receive an incoming notification. The instructions, when executed by one or more processors, may cause the agent to request, in response to the incoming notification, an input on a user interface, wherein the input includes a first input received from a first input type and a second input received from a second input type, wherein the first input type is different from the second input type.. The instructions, when executed by one or more processors, may cause the agent to determine the command based on the input from the user interface. The instructions, when executed by one or more processors, may cause the agent to cause an action to be performed based on the command.

In some embodiments, a computer-implemented method may comprise: receiving an electroencephalogram (EEG) dataset for training a classification model to determine a command type. The computer-implemented method may further comprise training the classification model using the EEG dataset. The computer-implemented method may further comprise receiving a first input comprising first EEG data from an EEG input component. The computer-implemented method may further comprise determining the command using the first EEG data.

The objects and advantages of the embodiments will be realized and achieved at least by the elements, features, and combinations particularly pointed out in the claims.

Both the foregoing general description and the following detailed description are given as examples and are explanatory and are not restrictive of the invention, as claimed.

### BRIEF DESCRIPTION OF THE DRAWINGS

Example embodiments will be described and explained with additional specificity and detail through the use of the accompanying drawings in which:

FIG. 1 illustrates an example virtual assist device configured to be wearable.

FIG. 2 illustrates an example process flow of a virtual assist device.

FIG. 3 illustrates an example process flow of a virtual assist device.

FIG. 4 illustrates an example process flow of a virtual assist device.

FIG. 5 illustrates an example process flow of a virtual assist device.

FIG. 6 illustrates an example process flow of a computer-readable storage medium including computer executable instructions for a virtual assist device.

FIG. 7 illustrates an example process flow for a computer implemented method for a virtual assist device.

FIG. 8 illustrates an example communication system for the virtual assist device.

FIG. 9 illustrates a diagrammatic representation of a machine in the example form of a computing device within which a set of instructions, for causing the machine to perform any one or more of the methods discussed herein, may be executed.

### DESCRIPTION OF EMBODIMENTS

The increase in communication has increased the number of notifications received from various devices. Monitoring the incoming notifications (e.g., email, texts, phone calls, and the like) uses a lot of time, and in many cases the notification may not be time-sensitive or result in action by a user.

An intelligent virtual assistant (IVA) or intelligent personal assistant (IPA) is a software agent that may perform tasks or services for an individual based on various information, including commands or questions. However, an IVA or IPA may not operate without adequate input from the individual.

Eliciting adequate information from a user to respond to a notification may not provide the time savings or reduced cognitive effort that an IVA or IPA desires to achieve. For example, upon receiving a text message, the user, just by glancing at the text message, has already consumed time and cognitive effort that interrupts their thought process. Additionally, unless the user provides input in response to the notification, the user may subsequently spend additional time determining whether the text message is time-sensitive or meaningful. Although filters may prevent some notifications from coming through (e.g., phone calls from unknown phone numbers), an additional set of notifications may not be filtered without additional input.

In addition, once a user has decided that a notification is time-sensitive and/or meaningful, processing the notification may use additional time or cognitive effort. For example, a person may receive a text message and determine from the content of the text message that a phone call to a particular person is time-sensitive. However, the process of deciding to call a particular person and initiating the call may use additional time and/or cognitive load. For a person with a large number of contacts, initiating the phone call may use the amount of time to locate a mobile phone, unlock the phone, scroll to a particular contact, and then initiating the call. For people who receive many calls, the collective amount of time saved may be substantial.

In some cases, a user may be in a public setting and may not desire to vocalize a command or response to a phone. For example, a user who is notified of a text message may not be able to respond to the text message by simply speaking out loud because such an action would interfere with others in the public setting. In some circumstances (e.g., during a meeting or during a lecture), a person may not be permitted to access a phone or respond to a message using a computerized assistant that relies on audible commands. In these circumstances, accessing and responding to information on a device without touching the device or vocalizing a command or response may allow the user to process the notification and respond to the notification in such settings.

Therefore, systems, devices, and methods for reducing the loss of time associated with filtering through notifications and responding to notifications is desirable.

In some embodiments, a virtual assist device may comprise an agent configured to receive an incoming notification and request an input in response to the incoming notification. The virtual assist device may comprise an electroencephalogram (EEG) input component configured to receive the input comprising EEG data and send the input to the agent. The agent may be configured to determine a command based on the input. A communication unit may be configured to cause an action to be performed based on the command.

Embodiments of the present disclosure will be explained with reference to the accompanying drawings.

In some embodiments, a virtual assist device may comprise an agent, an electroencephalogram (EEG) input component, and a communication unit. The agent may be implemented using machine-readable instructions as described herein and may be configured to receive an incoming notification. A notification may be any communication sent to a user interface to provide a user with a reminder (e.g., a badge, a banner, a user equipment (UE) notification), a communication from other people (e.g., a simple message service (SMS) message, an email, a phone call, or the like), or other time-sensitive information (an emergency alert, a meeting, an appointment, or a calendar request). The user interface may be a graphical user interface (e.g., a display on a UE), an auditory user interface (e.g., a headset), or the like.

In some embodiments, the agent may be configured to request an input in response to the incoming notification. The agent may be configured to notify the user of the incoming notification in one or more ways including, but not limited to: a sound (e.g., an audio chime, a spoken word), haptic feedback (e.g., vibration of the device or an accessory worn on the body), electrical stimulation, magnetic stimulation, visual stimulation (such as on a display mounted to the head or a display on a UE), or some combination of the aforementioned methods.

In some embodiments, the agent may be configured to notify the user of the content or some additional details of the notification such as “text message received from mom”, “email from boss,” “message within [name of app] from [name of friend].” The agent may be configured to request an action from the user, such as a reading of the notification (e.g., a reading of the text, the email, or other communication), a response to the notification (e.g., yes/no), or a time-sensitive labeling of the notification (e.g., emergency, urgent, important, redundant, spam, or the like). The agent may be configured to request user action through different ways (e.g., sound, haptic feedback, electrical stimulation, magnetic stimulation, visual stimulation, or the like).

In some embodiments, the agent may be configured to accept one or more inputs via a device (e.g., microphone, haptic input receiver, or a UE (e.g., touch screen, smartphone, cell phone, tablet, laptop, computer, or other computing device)). The input may be received by the agent in the form of clicking buttons or touching the touchscreen in order to perform tasks that may make the day-to-day life of a user easier.

In some embodiments, the agent may be configured to monitor a plurality of communication channels from different input components (e.g., microphone, UE, etc.). The agent may be configured to accept the one or more inputs from a first input component in a first time window and a second input component in a second time window in which the first time window and the second time window overlap (e.g., receiving a sound from the microphone at within a few seconds of receiving a touchscreen input from a UE). The agent may be configured to switch from the first input component to the second input component based on the data received by each input component (e.g., there may be no sound from the microphone and there may be input received from the touchscreen on the UE).

In some embodiments, the agent may be configured to receive both the first and second input data and cause an action to be performed based on the both sets of data. For example, the agent may receive first input data (via a spoken word) from a user comprising “left” in a first time window and second input data (via a touchscreen) from the user comprising “right” in a second time window. When the first and second input data is consistent (e.g., spoken “left” and touchscreen “left”), the agent may cause an action to be performed that is consistent both the first and second input data (“left”). When the first and second input data is not consistent (e.g., spoken “left” and touchscreen “right”), the agent may cause an action to be performed that is consistent with the input component that is computed as being more reliable based on a machine learning model computed using datasets from both input components (e.g., spoken “left” may be computed as being more reliable relative to touchscreen “right”).

In some embodiments, the agent may be configured to monitor any suitable number of channels from any suitable number of different input components in any number of suitable time windows to receive data that is suitable to cause an action to be performed. In some examples, a first input component may be an electroencephalogram (EEG) input component, a second input component may be a microphone input component, and a third input component may be an accelerometer. The first input data may be an EEG pattern associated with a “yes” as classified using a machine learning model. The second input data may be a sound associated with a “no” as classified using a machine learning model. The third input may be a movement pattern associated with a nod as classified using a machine learning model. The agent may be configured to cause an action to be performed based on the three different input components with each input component being weighted by a reliability metric. The reliability metric may be computed as a weighted average of the different input components or may be computed using a machine learning model that has been trained using datasets drawn from the different input components (or additional datasets drawn from input components that have not provided input data).

In some embodiments, the input component may comprise an EEG input component configured to receive input comprising EEG data and send the input to the agent. The input component may comprise a different input component, comprising input data that may be sent to the agent, including one or more of: a magnetoencephalography (MEG) input component, an electromyography (EMG) input component, an electrocardiogram (ECG) input component, or a photoplethysmography (PPG) input component, a microphone input component, a vibration sensor input component, an accelerometer input component, a capacitive input component, a resistive input component, a button click component, or the like.

In some embodiments, the EEG input component may comprise one or more sensors configured to contact a user at a selected cranial position to receive the EEG data used to determine the command. At least one of the sensors may be configured to contact a user at a cranial position at a language processing area of the brain. In one example, at least one of the sensors may be configured to contact a user at a cranial position at Wernicke’s area in the posterior superior temporal lobe (i.e., an area of the brain involved in language processing that is written or spoken and in language comprehension). In another example, at least one of the sensors may be configured to contact a user at a cranial position at Broca’s area in the left hemisphere (i.e., an area of the brain involved in speech production and articulation).

In some embodiments, the one or more sensors may be configured to contact a user at a cranial position at a language processing area of the brain and one or more additional cranial positions. The one or more additional cranial positions may not be at a language processing area of the brain.

In some embodiments, the sensors may be configured to extend from a housing for the input component (e.g., the EEG input component) to provide additional sensing points in selected areas on the user’s head, neck, or body. In one example, appendages may extend outward from the ear towards the temple or further behind the ear, to the forehead, top of the head, other side of the head, or any other part of the head, neck, or face where additional sensing inputs are desirable.

In some embodiments, other sensors may be used with the EEG input component including MEG sensors, EMG sensors, ECG sensors, or PPG sensors in contact with the user to function as percepts or sensors that provide data to the device about the user’s thoughts, feelings, emotions, desires, or other mental/physical states. In some examples, the sensors may be dry, semi-dry, wet sensors, the like, or a combination thereof. The sensors may be contacts such as pogo-pins that may contact the user’s skin. In some embodiments, the sensors may contact some portion of an ear of a user on one or more of the outer surface or the inside surface (including the ear canal).

In some embodiments, other sensors may be used with the EEG input component. In one example, an accelerometer may be used with the EEG input component. The accelerometer may provide movement data to the agent. The agent may be configured to use the movement data (e.g., associated with a head shake or head nod) to cause an action to be performed.

In some embodiments, the sensors may include one or more filters for the data input including, but not limited to: notch filters, high-pass filters, low-pass filters, band-pass filters, or the like. The one or more additional filters may be configured to remove any electrical noise or outside interference that may provide difficulty for sensing the input. The one or more filters may be configured to boost the signal, which may be a low voltage signal. The one or more filters may include, but are not limited to, any suitable analog front-end filter to filter a selected frequency range, band, or sub-band. The device may comprise one or more amplifiers to boost the signal, such as low-noise amplifiers (LNAs) or power amplifiers (PAs). The device may comprise one or more attenuators to attenuate a signal. The combination of the one or more filters, the one or more amplifiers, and the one or more attenuators may boost the signal as measured by a signal to noise ratio (SNR), a signal-to-noise plus interference ratio (SINR), or the like.

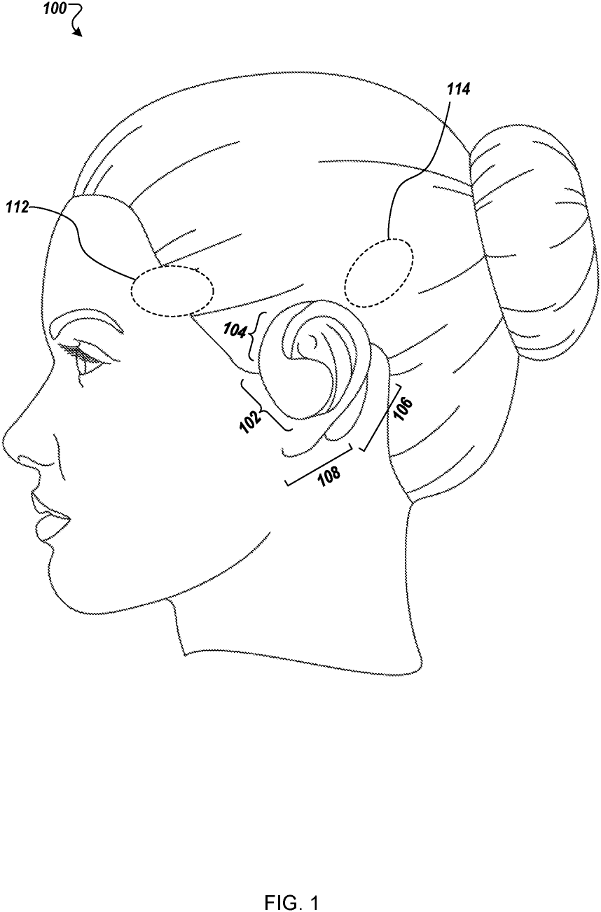

In some embodiments, the virtual assist device may comprise a wearable device housing the EEG input component, the communication unit, and the agent. As illustrated in FIG. 1, the wearable device 100 may comprise an earpiece 102, configured to provide sound to a user. The earpiece may be physically coupled to one or more portions of an appendage 104, 106 that may house one or more sensors (e.g., EEG sensors). The portion 104 of the appendage may be used to secure the earpiece within or near the ear canal and provide adequate contact to a portion of the brain to collect EEG input data (e.g., near Broca’s area). The portion 106 of the appendage may be used to secure the earpiece around the back of the ear 108 of the user and provide adequate contact to a different portion of the brain to collect EEG input data (e.g., Wernicke’s area). The appendage having portions 104, 106 may curl around the ear 108 of a user to contact one or more of Broca’s area 112 or Wernicke’s area 114 to receive EEG input data from a region of the brain involved in language comprehension and formation. The wearable device 100 may further comprise a microphone configured to receive auditory input from a user.

In some embodiments, the wearable may be an over-ear, on-ear, or in-ear device with a battery, microphone(s), speaker, volume rocker, yes/no/[other function] buttons, accelerometer, capacitive or resistive touch sensors, Bluetooth™ or other wireless connectivity (e.g., WiFi, mmWave, 3GPP, etc.), or vibration motor (linear, rotary, or combination of the two).

Although a specific example of a wearable has been provided, the virtual assist device may further comprise any device disclosed herein, such as a UE (e.g., a mobile device, portable device, wearable device, smartphone, tablet, computer, sticker computer, or any other computing device).

In some embodiments, the virtual assist device may comprise an electromagnetic shield for preventing interference between the one or more input signals from the different input types and external interference sources. The electromagnetic shield may comprise any suitable conductive or metallic material used for shielding an interfering electromagnetic source such as sheet metal, a metal screen, or a metal foam comprising one or more of copper, brass, nickel, silver, steel, tin, or the like.

In some embodiments, an electromagnetic shield may prevent interference with an EEG input component. Measuring EEG signals through a cranium involves signals that may have a frequency from about 1 Hertz (Hz) and about 30 Hz (e.g., a frequency of about 1 Hz to about 3 Hz for delta waves, a frequency of about 3 Hz to about 7.5 Hz for theta waves; a frequency of about 7.5 Hz to about 13 Hz for alpha waves; and a frequency of about 14 Hz to about 30 Hz for beta waves; greater than about 31 Hz for gamma waves), and an amplitude of from about 2 µV to about 100 µV (20-100 µV for delta waves; about 10 µV for theta waves; 2-100 µV for alpha waves; 5-10 µV for beta waves; 20-100 µV for delta waves; and a varying amplitude for gamma waves). Alternating current (AC) electrical sources (e.g., from electrical lines and devices) may interfere with the low amplitude signals from an EEG. The AC electrical sources may superimpose a 50 to 60 Hz electrical artifact overlapping the EEG signal. An electromagnetic shield may be used to prevent artifacts caused by these AC electrical sources.

In some embodiments, the agent may be configured to determine a command based on the input data. The input data may be received from one or more input components. When more than one input component is used to send input data, the input data may be received from different input component types (e.g., an EEG and a touchscreen). In some embodiments, the agent may be configured to determine a command based on one or more additional input types. In one example, when EEG input data is received, the input data may further include second input data and third input data from a second input component (e.g., from a touchscreen) and a third input component (e.g., from a microphone) in which the second and third input components are different input components. The agent may be further configured to determine the command using one or more of the second input data (e.g., data from the touchscreen) or the third input data (e.g., data from the microphone).

In some embodiments, the command may be a “yes” command or a “no” command. The input data may be determined as a “yes” command or “no” command in various ways including but not limited to: (i) a spoken response, (ii) clicking dedicated buttons or other-use buttons on the device or accessory (e.g. volume buttons as other-use buttons, and yes/no buttons on the communication device as dedicated buttons), (iii) tapping a device that contains a physical sensor (e.g., an accelerometer, capacitive sensor, resistive sensor, or some other sensor for detecting physical input); (iv) head nodding, head movement, or body movement that may be detected by movement sensors such as an accelerometer; (v) audibly speaking the command which may be heard by the device or accessory via a microphone or inaudibly mouthing the command which may be sensed by a vibration sensor (e.g. bone conduction), or (vi) thinking the command which may be sensed using a sensor coupled to an EEG input component.

In some embodiments, the agent may be configured to determine the command using a mapping between a particular input and a “yes” command or a “no” command. In some examples, a particular hand gesture (e.g., horizontal swiping on a touch screen) or tap pattern (tapping once) may be mapped to a “no” response and a particular hand gesture (vertically swiping on a touch screen) or tap pattern (tapping twice) may be mapped to a “yes” response.

In some embodiments, the agent and/or the communication unit may be configured to cause an action to be performed based on the command. The action may include one or more of: (i) communicating the notification to a user interface (e.g., reading the notification in audio form, displaying the notification on a graphical display on a UE, providing haptic feedback associated with the notification, or the like), (ii) requesting an additional command, (iii) requesting an outgoing notification, (iv) communicating an outgoing notification, or (v) ending a response request.

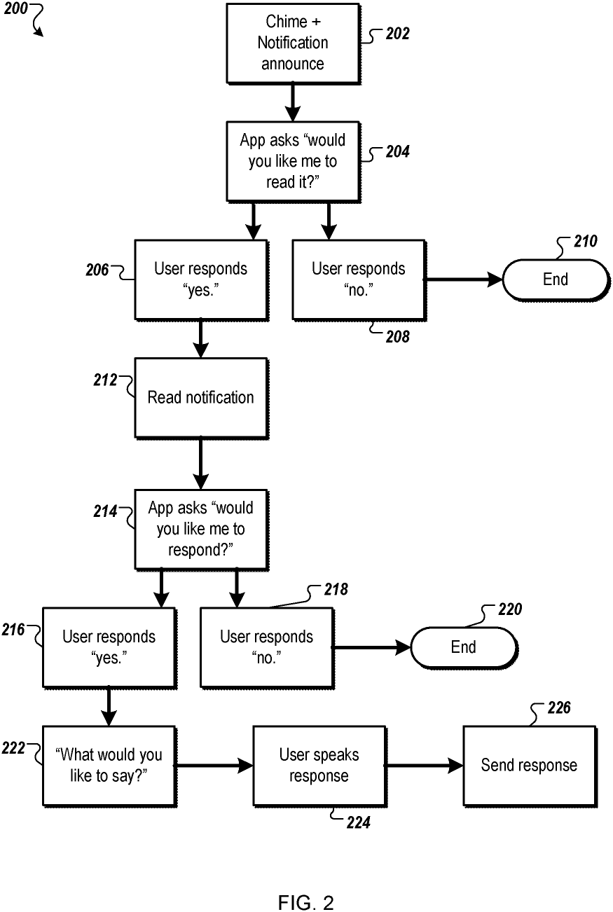

In some embodiments, the agent may be configured to cause an action to be performed based on one or more of a “yes” command or a “no” command, as illustrated in FIG. 2 with respect to the functionality 200 of an App on a user equipment. When the App receives an incoming notification, the App may be configured to request an input in response to the incoming notification by announcing the notification using a chime, as in operation 202. The App may be configured to ask “Would you like me to read it?” as shown in operation 204. When the App receives a “no” response from the user, as shown in operation 208, the App may terminate the response request, as shown in operation 210. When the App receives a “yes” response, as shown in operation 206, the App may cause an action to be performed by communicating the notification to the user interface (e.g., read the notification), as shown in operation 212. After reading the notification, the App may ask “would you like me to respond?” as shown in operation 214. When the App receives a “yes” response, as shown in operation 216, the App may cause an action to be performed by requesting an outgoing notification (e.g., ask “What would you like to say?”), as shown in operation 222. When the App receives a response from the user (e.g., user speaks response), as shown in operation 224, the App may cause an action to be performed by communicating an outgoing notification (e.g., send response), as shown in operation 226.

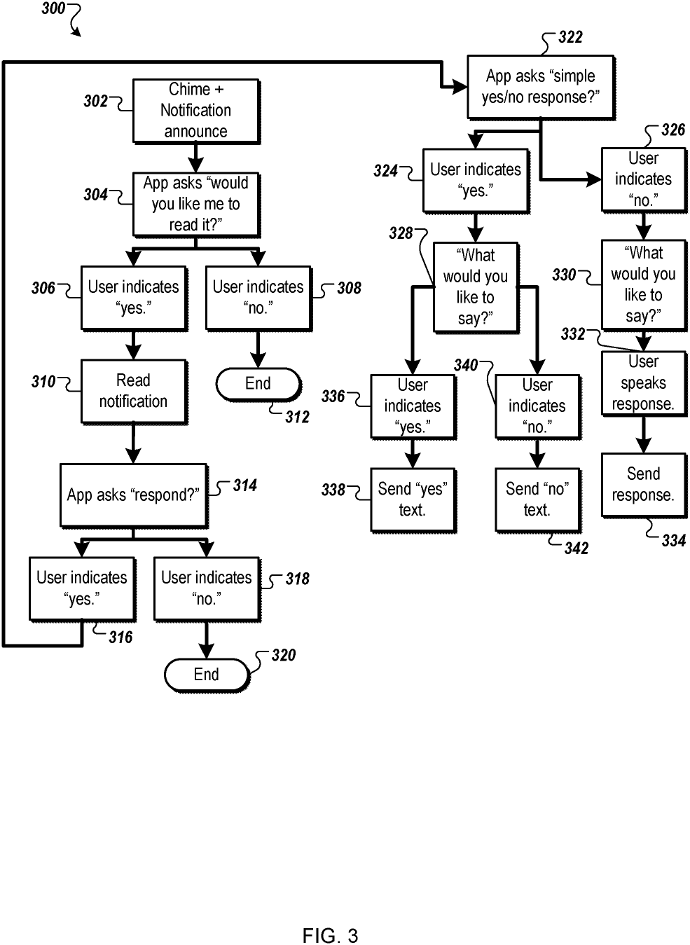

In some embodiments, the agent and/or the communication unit may be configured to cause an action to be performed based on one or more of a “yes” command or a “no” command, as illustrated in FIG. 3 with respect to the functionality 300 of an App and a device. The App may be configured similarly, as described with respect to FIG. 2 for operations 202, 204, 206, 208, 210, 212, 214, 216, 218, 220, with respect to operations 302, 304, 306, 308, 310, 312, 314, 316, 318, and 320, respectively. When the App receives a “yes” response, as shown in operation 320, the App may cause an action to be performed by requesting an additional command (e.g., App asks “simple yes/no response?”), as shown in operation 322. When the App receives a “no” response, as shown in operation 326, the App may cause an action to be performed by requesting an outgoing message (e.g., “what would you like to say?”), as shown in operation 330. In response to the user providing a spoken response, as shown in operation 332, the App may cause an action to be performed by communicating an outgoing notification (e.g., send response), as shown in operation 334. When the App receives a “yes” response, as shown in operation 324, the App may be cause an action to be performed by requesting an outgoing message (e.g., “what would you like to say?”), as shown in operation 328. When the user indicates “no”, as shown in operation 340, the App may cause an action to be performed by communicating an outgoing notification (e.g., send “no″ text”), as shown in operation 342. When the user indicates “yes, as shown in operation 336, the App may cause an action to be performed by communicating an outgoing notification (e.g., send “yes“ text“), as shown in operation 338.

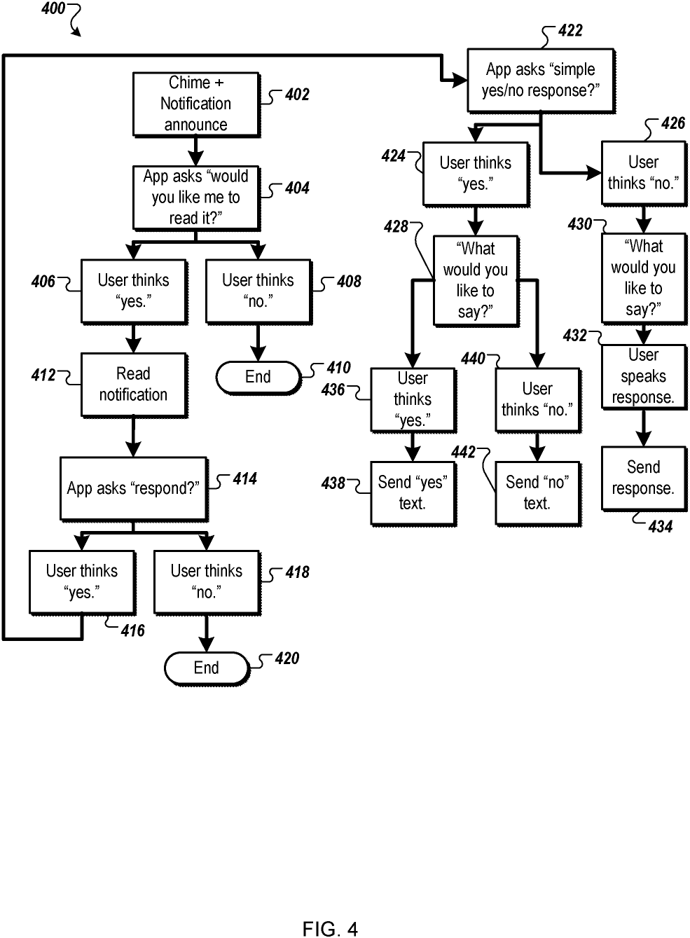

In some embodiments, the agent and/or the communication unit may be configured to cause an action to be performed based on one or more of a “yes” command or a “no” command, as illustrated in FIG. 4 with respect to the functionality 400 of an App, an EEG input component, and a communication unit. Thus, the device may include an EEG input component that may receive and/or interpret brain activity, such as by detecting electrical activity in the brain, where a “yes” response is represented by a particular and distinct brain activity and/or brain wave, and a “no” response may be represented by a different particular and distinct brain activity and/or brain wave, such that the EEG may distinguish between a “yes” and a “no” response based on data received and/or detected by the EEG.

The App may be configured similarly, as described with respect to FIG. 3 for operations 302, 304, 310, 312, 314, 320, 322, 328, 330, 332, 334, 338, 342, with respect to operations 402, 404, 406, 408, 410, 412, 414, 420, 422, 428, 430, 432, 434, 438, 442, respectively. With respect to operations 406, 416, 424, 436 (“User thinks ‘yes.”’) and 408, 418, 426, and 440 (“User thinks ‘no.”’), the EEG input component may be configured to receive the input comprising EEG data and sent the input to the agent. The agent may be configured to determine a command (e.g., a “yes” command, as shown in operation 406, 416, 424, or a “no” command, as shown in operation 408, 418, 426) or a notification (e.g., a “yes” response, as shown in operation 436, or a “no” response, as shown in operation 442) based on the input. Thus, the virtual assist device may use these “yes” and “no” commands and responses for various actions. A user who is wearing a device with an EEG component may “think” their response to an external stimulus (e.g., a notification) and the virtual assist device may provide an appropriate response by taking an action.

In some embodiments, the virtual assist device may include a set of discrete wearable EEG sensors (e.g., capacitive sensors) embedded within a communication unit (e.g. Bluetooth® headset, earbuds, etc.) that may be configured to: (i) detect electrical activity of the brain (e.g., the µvoltage generated by neurons firing) through the skull, (ii) digitally filter these EEG signals to remove noise, and (iii) pass the filtered EEG signals to the device (e.g., UE), which may (iv) use a machine learning model (pre-trained e.g., hundreds of thousands or millions of other data points) to determine the command (e.g., “yes,” “no,” “left,” “right,” “up,” “down,” “option 1”, “option 2”, or the like) facilitating control of the UE and a quick and discrete way of parsing through incoming notifications. In some examples, the EEG sensors may comprise one or more of dry, semi-dry, or wet sensors.

In some embodiments, an agent may be configured to determine a command by training a classification model using various datasets. The agent may be configured to receive an EEG dataset for training the classification model to determine a command type. The agent may be configured to train the classification model using the EEG dataset. The agent may be configured to receive a second input dataset for training the classification model to determine the command type. In one example, the second input dataset may not be EEG data. The agent may be configured to train the classification model using the second input dataset.

In some embodiments, the classification model may be trained by using datasets that differ not only based on input type but also based on a user-specific condition and/or an environmental condition. In one example, the EEG dataset may be based on a user-specific condition or environmental conditions such as: (a) users thinking “yes” or “no” during various stages of drowsiness, (b) users skiing downhill at 60 mph in cold weather, (b) users sitting on a couch and watching TV, (d) users driving, (e) users exercising, (f) users at a concert, or the like. The classification model may be trained using a wide-ranging number of differing user-specific and environmental conditions to discern a pattern in the EEG data that is less subject to variation.

In some embodiments, a primary language of users in an EEG dataset may impact the training of a classification model. For example, a dataset that has been drawn from primary English speakers may differ from a dataset that has been drawn from primary German speakers. The dataset used for training the classification model may differ based on user characteristics such as primary language. The agent may be configured to determine a primary language or other user characteristics based on the EEG input.

In some embodiments, the virtual assist device may be initialized using various inputs, such as via a user interface. In one example, the user interface may be a touchscreen operating on an App on a UE. The touchscreen may display an input request by displaying, “After the first chime, close your eyes. After the second chime, open your eyes (5 seconds).” This input request may allow the agent to remove artifacts from EEG input data. In this example, eye movement artifacts may be captured so that these artifacts may be removed from the EEG input data to be received.

Other examples may be used to request input that may be used to remove artifacts from EEG input data including one or more of: requesting a body movement (e.g., movement of arms, hands, fingers, legs, head, feet, facial movements, such as frowning, talking, chewing, jaw clenching, neck/shoulder tension, swallowing, sniffing, grimacing, or any other portion of the body), requesting an action to adjust a heart rate; requesting an action to adjust an amount of perspiration; requesting an action to adjust an amount of respiration; or otherwise requesting an action that may adjust a physiological property to provide a baseline for removing artifacts from input data.

In some embodiments, the user interface may request input that may be used to train a model. The user interface may display an input request (“After the chime, think the word ‘Yes″’) to request a user to think of a particular command (e.g., an affirmative command). The user interface may display an input request (“After the chime, say the word ‘Yes’ in your head) to request a user to think of a particular command (e.g., an affirmative command) in a way that includes an additional type of thinking (e.g., saying in your head). The user interface may display an input request (“After the chime, say the word ‘Yes‘ out loud”) to request a user to think of a particular command (e.g., an affirmative command) in a way that includes an additional type of thinking (e.g., saying in your head) and an additional action (talking).

In some embodiments, the user interface may request input that may be subtractive or additive to other requested input. For example, the user interface may request various additional ways of thinking a particular command (e.g., picturing, “After the chime, picture the word ‘Yes’”). The user interface may request input that includes a perception (e.g., “After the chime, stare at the word below” in which the word below graphically displays the word “yes”). A difference between thinking of a particular command and perceiving a particular command may be used to train the model by removing noise. This difference between thinking of a particular command, perceiving a particular command, and saying a particular command may be further combined to allow for the subtraction of noise (e.g., “After the chime, stare at the word below while saying “yes” in your head” in which the word “YES” is graphically displayed below).

In some embodiments, the user interface may request input to train a machine learning model to distinguish between “yes” and “no.” The user interface may display an input request (“think ‘yes’ or ‘no‘ and confirm that the appropriate circle turns green”) to test whether the agent may distinguish between an affirmative command and a negative command when the type of command is not requested. The user interface may be configured to display two options to request user input to confirm that the ‘yes’ or ‘no’ response was correctly determined by the agent: (1) “It worked!” and (2) “Train Again.” When the ‘yes’ or ‘no’ response is correctly recorded, the user interface may display an additional initialization screen. When the ‘yes’ or ‘no’ response is not correctly recorded, the user interface may re-display the same training screen to request the input again. The process may continue for the command that was not initialized (i.e., “no” may be initialized when “yes” has been initialized and vice versa).

In some embodiments, the user interface may request input to train a machine learning model to distinguish between a rapid succession of patterns of “yes” commands and “no” commands. For example, the user interface may request a “yes” command and request confirmation that the “yes” command was correctly determined. The user interface may then request another “yes” command or a “no” command and further request confirmation that the command was correctly determined.

In some embodiments, the agent may be configured to request the type of notifications to be notified about during the initialization process. The agent may request notification settings with respect to, e.g., phone calls from contacts or everyone, text messages from contacts or everyone, group texts from contacts or everyone, emails from contacts or everyone, and calendar invites from contacts or everyone. The notifications may be configured to be received from any selectable subset of people (e.g., favorites, close contacts, contacts, contacts plus others within a specific distance).

In some embodiments, during initialization, the agent may be configured to request any additional input that may be used to train the machine learning model. For example, the agent may be configured to request one or more of: speaking, nodding, or tapping, while thinking a command or not thinking a command. The agent may further be configured to request any additional input from a non-EEG source (e.g., EMG input) that may be used to complement the input data from the EEG to further refine the training of the model. The model may be trained using various types of input to personalize the determination of the command for a particular user.

In some embodiments, the EEG input data may be pre-processed to remove artifacts. Artifacts may include physiological artifacts (e.g., ocular, muscle, cardiac, perspiration, or respiratory) or non-physiological (e.g., electrode pop, cable movement, incorrect reference placement, AC EM interference, or body movements). Pre-processing the EEG input data to remove these artifacts may enhance the accuracy in determining the EEG input data as a command.

In some embodiments, the agent may be configured to receive an input comprising EEG data from an EEG input component, and one or more additional input data from a one or more additional input components. The one or more additional input data may not be EEG data and may be received from non-EEG components. The non-EEG input components configured to send non-EEG data may include one or more of: an MEG input component, an EMG input component, an ECG input component, a PPG input component, a microphone input component, a vibration sensor input component, an accelerometer input component, a capacitive input component, a resistive input component, a button click component, or the like.

In some embodiments, the agent may be configured to determine the command using one or more of: the EEG data and the one or more additional input data. In some embodiments, the agent may be configured to determine the command by: training a model using training input comprising an EEG dataset, and identifying the command using the model. In some embodiments, the model may be a classification model. In some embodiments, the model may be one or more of: a convolutional neural network (e.g., having a suitable number of dimensions such as 1-dimensional, 2-dimensional, and so forth), a long term short memory network, a recurrent neural network, a sequence to sequence model, a transformer model, or the like.

In some embodiments, the classification model may involve binary classification, multi-class classification, multi-label classification, or the like. Binary classification may use a model used to predict a Bernoulli probability distribution and may be computed using one or more of logistic regression, k-nearest neighbor computation, a decision tree, a support vector machine, a naïve Bayes computation, or the like. Multi-class classification may use a model used to predict a categorical distribution and may be computed using one or more of k-nearest neighbor computation, a decision tree, a naive Bayes computation, a random forest computation, or gradient boosting. In some examples, a binary classification computation may be used to perform a multi-class classification by fitting multiple binary classifications and may be computed using logistic regression and/or a support vector machine. Multi-label classification may use a model to predict a Bernoulli distribution for multiple outputs and may be computed using a multi-label decision tree computation, a multi-label random forest computation, or a multi-label gradient boosting computation.

In some embodiments, a convolutional neural network may comprise an input layer, an output layer, and one or more convolutional layers. The input layer may comprise a tensor comprising a number of inputs, an input height, an input width, and input channels. The inputs may be passed through a convolutional layer (e.g., by computing a dot product between the input layer and kernels) to form an activation map having a shape comprising a number of inputs, a feature map height, a feature map width, and feature map channels. The feature map may be further processed in one or more pooling layers (e.g., using local pooling, global pooling, max pooling, and/or average pooling), one or more fully-connected layers, and one or more normalization layers. The size of the output volume may be configured based on the depth, stride, and padding size. The memory footprint and/or computational complexity may be configured based on the type of pooling used. Other parameters, such as the number of kernels, the kernel size, the pooling size, and/or the dilation may be used to determine a command with an acceptable margin of error.

In some embodiments, a recurrent neural network may use previous outputs as inputs. The recurrent neural network may comprise, for each time-step, an activation expression and an output expression. The activation may comprise: a(t) = g₁ (Wₐₐ a(t-1) -Wₐₓ(t) + bₐ) and the output expression may comprise: y(t) = g₂(W_{ya} a(t) + b_{y}), wherein Wₐₓ, Wₐₐ, W_{ya}, bₐ, and b_{y} are coefficients at the same time-step, and g₁, g₂ are activation functions. In some examples, the activation function may be one or more of a sigmoid function, a tanh function, or a rectified linear unit (ReLU) function. Gradient explosion may be reduced by using gradient clipping and gradient vanishing may be reduced by using one or more gates (e.g., an update gate, a relevance gate, a forget gate, or an output gate).

In some embodiments, long term short memory network may comprise one or more long short-term memory units including a cell, an input gate, and output gate, and a forget gate. The forget gate may be configure to discard information from a previous state, an input gate may be configured to store information from a previous state, and output gate may be configured to determine the information to output. The information (e.g., digital data) may flow between the different gates between a plurality of cells.

In some embodiments, the sequence to sequence model (S2S) model may be configured to use a recurrent neural network to: encode an input sequence (e.g., an input word) into a vector including the sequence (e.g., an encoded word) and the sequence context, and decode the vector into an output sequence (e.g., an output word). In some examples, the S2S model may use one or more of: attention processing (the input may be a vector including the context and the decoder selects from the context), beam searching (the output may be structured as a tree of different selections in which each selection may be weighted), bucketing (to specify input and output lengths). Training the S2S model may use a cross-entropy loss function.

In some embodiments, the transformer model may be configured to use an encoder comprising a self-attention mechanism and a feed-forward neural network and a decoder comprising a self-attention mechanism, an attention mechanism for the encodings, and a feed-forward neural network. The encoder may be configured to generate encodings having contextual information. The decoder may be configured to generate an output sequence based on the encodings and the contextual information. The decoder may use an attention mechanism to receive information from the outputs of previous decoders before determining information from the encodings.

In some embodiments, the virtual assist device may use a machine learning model in order to analyze the signals from devices or accessories used for input. The machine learning model may be present on the virtual assist device, on a UE, or any other UE that may be paired with the virtual assist device. In the case of Electroencephalogram (EEG) input, the virtual assist device may use the model to differentiate thoughts or other inputs from the user. The model may determine what the user is doing in terms of thoughts, feelings, emotions, desires, or other mental/physical states.

In some embodiments, the agent may be configured to determine the command using one or more of the models disclosed herein by preprocessing an input data set (e.g., an EEG data set), removing artifacts from the input data set (e.g., EEG data), training a model using a training dataset as disclosed herein, and inputting the input data set (e.g., EEG data) into the model to distinguish between input data (e.g., EEG data) associated with a command and input data (e.g., EEG data) associated with noise.

In some embodiments, the command may be one or more of: an affirmative command, a negative command, a word, a numbered option, a directional option, an assistance-activating command, or a password for authenticating a user. An affirmative command may include a “yes” command. A “yes” command may be identified by the agent and the agent may cause an action to be performed based on the “yes” command such as reading an inbound notification or communicating an outbound notification. A negative command may include a “no” command that may be identified by the agent, which may cause an action to be performed based on the negative command (e.g., delaying a reading of an inbound notification or delaying or bypassing a response to an inbound notification).

In some embodiments, the command may be a word. The word may activate a plurality of actions comprised of component actions. For example, a word may be “home” which may be used to auto-fill a home mailing address (the component actions would include the series of words and numbers included in the home mailing address), or “work” which may be used to auto-fill a work mailing address, a “phone” which may be used to auto-fill a phone number, a specific contact which may activate actions specific to that contact.

In some embodiments, the command may be a numbered option. An agent may be able to determine a numbered option (e.g., option 1, option 2, option 3, and so forth) in which each numbered option may activate a plurality of actions associated with each numbered option. For example, in response to sending a notification to a user interface, option 1 may be used to send outgoing response text 1, option 2 may be used to send outgoing response text 2, option 3 may be used to send outgoing response text 3, and so forth. In some examples, each option may be associated with a letter, and a response may be generated by the agent by determining a series of numbered options and associating each of those numbered options with a letter to form words, sentences, and paragraphs.

In some embodiments, the command may be a directional option (e.g., “up”, “down”, “left”, “right”) in which each directional option may be associated with an action. For example, in determining a command to respond to a notification, “up” may be associated with “yes,” “down” may be associated with “no”, “left” may be associated with a one hour reminder, and “right” may be associated with a reminder the next day.

In some embodiments, the command may facilitate user authentication via a password. The command may activate user authentication when the agent identifies the password, using a model, as EEG input data corresponding to one or a memory, an response to audio stimulation (e.g., a specific song, chime, voice of a person), visual stimulation (a picture, a photo of a person), or the like.

In some embodiments, the command may comprise an assistance-activating command. The assistance-activating command may be configured to activate communication between a user interface and a virtual assistant. The agent may be configured to identify the assistance-activating command receiving from the user interface, and the agent may be configured to cause an action to be performed (e.g., communication between a user interface and a virtual assistant). The assistance-activating command may comprise a spoken word or phrase (e.g., “activate assistant”) that may be adjusted based on user preference. The assistance-activating command may comprise a thought (e.g., “I want to communicate with my virtual assistant”). The assistance-activating command may activate the virtual assistant when the agent identifies the assistance-activating command, using a model, as EEG input data corresponding to the assistance-activating command.

In some embodiments, the agent may be configured to actively filter incoming notifications (e.g., text messages, emails, social media, calendar, and the like) and may be trained over time using a model and based on usage data to determine which notifications to communicate via a user interface (e.g., attracting the attention of a user through a pleasant chime in the communication device which a user may respond to by thinking “yes” if they wish to hear what the notification is and “no” if they wish to ignore the notification).

In some embodiments, the agent may be configured to determine which notifications to filter by determining a notification type for the incoming notification, determining a notification time for the incoming notification based on the notification type; and requesting input on the user interface based on the notification time. In some examples, the agent may determine the command in less than a threshold amount of time after receiving the input from the user interface, wherein the threshold amount of time is less than one or more of: 1 minute, 30 s, 5 s, 1 s, 1 ms, 100 µs, 10 µs, or 1 µs. The notification type may be a classification provided to a notification that facilitates the filtering of the notification and the computation of the notification time.

In some embodiments, the agent may be configured to determine the notification type for the incoming notification by: training a model to determine the notification type using a training data-set comprising usage data. The usage data may comprise usage data collected from a representative number of different users and may further include usage data that is specific to a particular user. The agent may be configured to identify the notification type by using the model.

In some embodiments, the agent may be configured to determine a notification time for the incoming notification based on the notification type and based on a model that has been trained using usage data from a representative number of users and usage data that is specific to a particular user. The notification time is the time between the receipt of the notification by the agent and the time at which the notification is communicated to a user interface.

In some embodiments, the agent may be configured to request input on the user input based on the notification time. In some examples, the notification time may be further adjusted based on a non-EEG input component. For example, a communication device may include a microphone that may communicate to the agent that the microphone is in use and therefore a notification may not be provided to a user interface at a particular time. In another example, a calendar event may provide an indication to the agent that the agent may not provide a notification to the user interface during the calendar event (because the user may be in a meeting and therefore should not be interrupted.

In some embodiments, the virtual assist device may comprise a UE including a computer readable medium comprising a set of instructions, for causing the UE to perform any one or more of the methods discussed herein, may be executed on one or more processors.

In some embodiments, the virtual assist device may comprise a wearable device housing the EEG input component (or different or additional input components), the communication unit, and the agent. The wearable device may comprise a headset such as headphones, earbuds, or the like. In some embodiments, the wearable device (e.g., a headset) may be configured to process data that is received or the wearable device may rely on another device (e.g. a UE, or another device with a processor). The wearable device may allow the user to hear audio, music, prompts, voice commands, or any other audible sound. The wearable device may comprise one or more microphones to receive and process audio input from the user, surroundings, or any other source in an audible range. One or more microphones may be used for noise cancellation.

In some embodiments, the agent may be configured to receive EEG input data from an EEG input component and EMG input data from an EMG input component. The EEG input data may be used to train a model and the EMG data may be used to refine the model. In some cases, the wearable device may be comprise a virtual reality (VR) headset, an augmented reality (AR) headset, or a mixed reality (MR) headset. The agent may be configured to use the EEG input data to determine a command in the presence of movement from the user (e.g., as captured by the EMG input data). Furthermore, the EMG input data may be used to determine a command or response by the user in the VR or AR environment. In one example, a user interface in a VR or AR environment may notify a user about a notification based on the functionality of the agent as disclosed herein (e.g., by determining a command to display a notification, determining a command to ignore a notification, determining a command to respond to a notification, or the like).

The virtual assist device may be configured to have a charging input via inductive charging coils of various shapes (e.g., circular, oblong, square, triangular, or the like), exposed surface contacts that may or may not have special coating or plating using platinum-group metals to prevent deterioration from environmental sources (such as liquid, heat, sun, sweat, or the like). In another example, the virtual assist device may have may be charged using a wired connection (e.g., a USB connection).

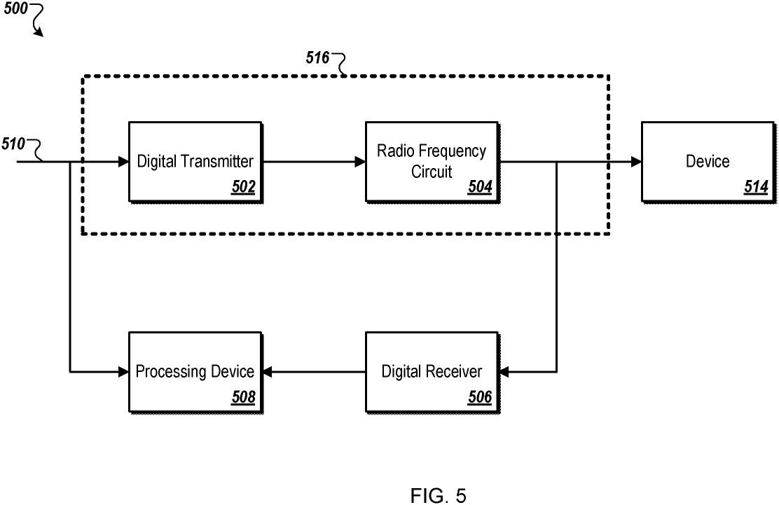

The communication unit may be configured as shown with respect to the communication system of FIG. 5, which illustrates a block diagram of an example communication system 500 configured for communicating inbound and outbound notifications, in accordance with at least one embodiment described in the present disclosure.

The communication system 500 may include a digital transmitter 502, a radio frequency circuit 504, a device 514, a digital receiver 506, and a processing device 508. The digital transmitter 506 and the processing device may be configured to receive a baseband signal via connection 510. A transceiver 516 may comprise the digital transmitter 502 and the radio frequency circuit 504.

In some embodiments, the communication system 500 may include a system of devices that may be configured to communicate with one another via a wired or wireline connection. For example, a wired connection in the communication system 500 may include one or more Ethernet cables, one or more fiber-optic cables, and/or other similar wired communication mediums. Alternatively, or additionally, the communication system 500 may include a system of devices that may be configured to communicate via one or more wireless connections. For example, the communication system 500 may include one or more devices configured to transmit and/or receive radio waves, microwaves, ultrasonic waves, optical waves, electromagnetic induction, and/or similar wireless communications. Alternatively, or additionally, the communication system 500 may include combinations of wireless and/or wired connections. In these and other embodiments, the communication system 500 may include one or more devices that may be configured to obtain a baseband signal, perform one or more operations to the baseband signal to generate a modified baseband signal, and transmit the modified baseband signal, such as to one or more loads.

In some embodiments, the communication system 500 may include one or more communication channels that may communicatively couple systems and/or devices included in the communication system 500. For example, the transceiver 516 may be communicatively coupled to the device 514.

In some embodiments, the transceiver 516 may be configured to obtain a baseband signal. For example, as described herein, the transceiver 516 may be configured to generate a baseband signal and/or receive a baseband signal from another device. In some embodiments, the transceiver 516 may be configured to transmit the baseband signal. For example, upon obtaining the baseband signal, the transceiver 516 may be configured to transmit the baseband signal to a separate device, such as the device 514. Alternatively, or additionally, the transceiver 516 may be configured to modify, condition, and/or transform the baseband signal in advance of transmitting the baseband signal. For example, the transceiver 516 may include a quadrature up-converter and/or a digital to analog converter (DAC) that may be configured to modify the baseband signal. Alternatively, or additionally, the transceiver 516 may include a direct radio frequency (RF) sampling converter that may be configured to modify the baseband signal.

In some embodiments, the digital transmitter 502 may be configured to obtain a baseband signal via connection 510. In some embodiments, the digital transmitter 502 may be configured to up-convert the baseband signal. For example, the digital transmitter 502 may include a quadrature up-converter to apply to the baseband signal. In some embodiments, the digital transmitter 502 may include an integrated digital to analog converter (DAC). The DAC may convert the baseband signal to an analog signal, or a continuous time signal. In some embodiments, the DAC architecture may include a direct RF sampling DAC. In some embodiments, the DAC may be a separate element from the digital transmitter 502.

In some embodiments, the transceiver 516 may include one or more subcomponents that may be used in preparing the baseband signal and/or transmitting the baseband signal. For example, the transceiver 516 may include an RF front end (e.g., in a wireless environment) which may include a power amplifier (PA), a digital transmitter (e.g., 502), a digital front end, an Institute of Electrical and Electronics Engineers (IEEE) 1588v2 device, a Long-Term Evolution (LTE) physical layer (L-PHY), an (S-plane) device, a management plane (M-plane) device, an Ethernet media access control (MAC)/personal communications service (PCS), a resource controller/scheduler, and the like. In some embodiments, a radio (e.g., a radio frequency circuit 504) of the transceiver 516 may be synchronized with the resource controller via the S-plane device, which may contribute to high-accuracy timing with respect to a reference clock.

In some embodiments, the transceiver 516 may be configured to obtain the baseband signal for transmission. For example, the transceiver 516 may receive the baseband signal from a separate device, such as a signal generator. For example, the baseband signal may come from a transducer configured to convert a variable into an electrical signal, such as an audio signal output of a microphone picking up a speaker’s voice. Alternatively, or additionally, the transceiver 516 may be configured to generate a baseband signal for transmission. In these and other embodiments, the transceiver 516 may be configured to transmit the baseband signal to another device, such as the device 514.

In some embodiments, the device 516 may be configured to receive a transmission from the transceiver 516. For example, the transceiver 516 may be configured to transmit a baseband signal to the device 514.

In some embodiments, the radio frequency circuit 504 may be configured to transmit the digital signal received from the digital transmitter 502. In some embodiments, the radio frequency circuit 504 may be configured to transmit the digital signal to the device 514 and/or the digital receiver 506. In some embodiments, the digital receiver 518 may be configured to receive a digital signal from the RF circuit and/or send a digital signal to the processing device 508.

In some embodiments, the processing device 508 may be a standalone device or system, as illustrated. Alternatively, or additionally, the processing device 508 may be a component of another device and/or system. For example, in some embodiments, the processing device 508 may be included in the transceiver 516. In instances in which the processing device 508 is a standalone device or system, the processing device 508 may be configured to communicate with additional devices and/or systems remote from the processing device 508, such as the transceiver 516 and/or the device 514. For example, the processing device 508 may be configured to send and/or receive transmissions from the transceiver 516 and/or the device 514. In some embodiments, the processing device 508 may be combined with other elements of the communication system 500.

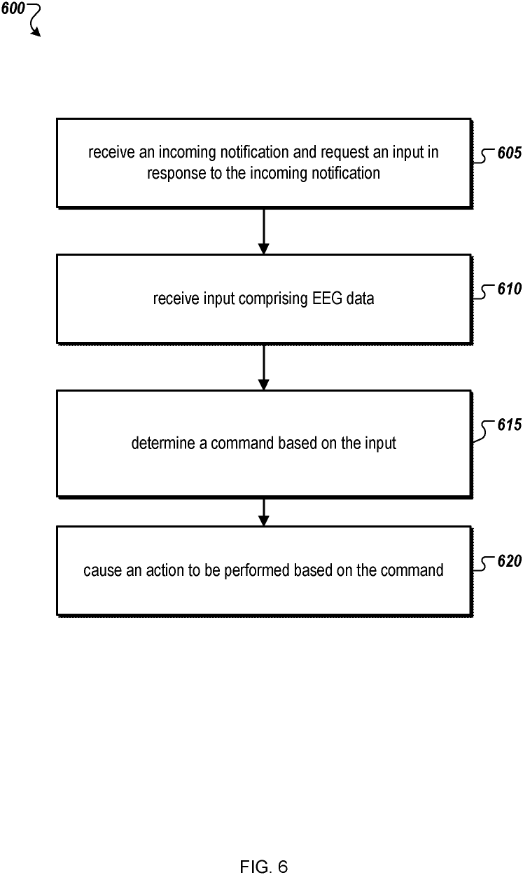

FIG. 6 illustrates a process flow of an example method 600 of virtual assist device, in accordance with at least one embodiment described in the present disclosure. The method 600 may be arranged in accordance with at least one embodiment described in the present disclosure.

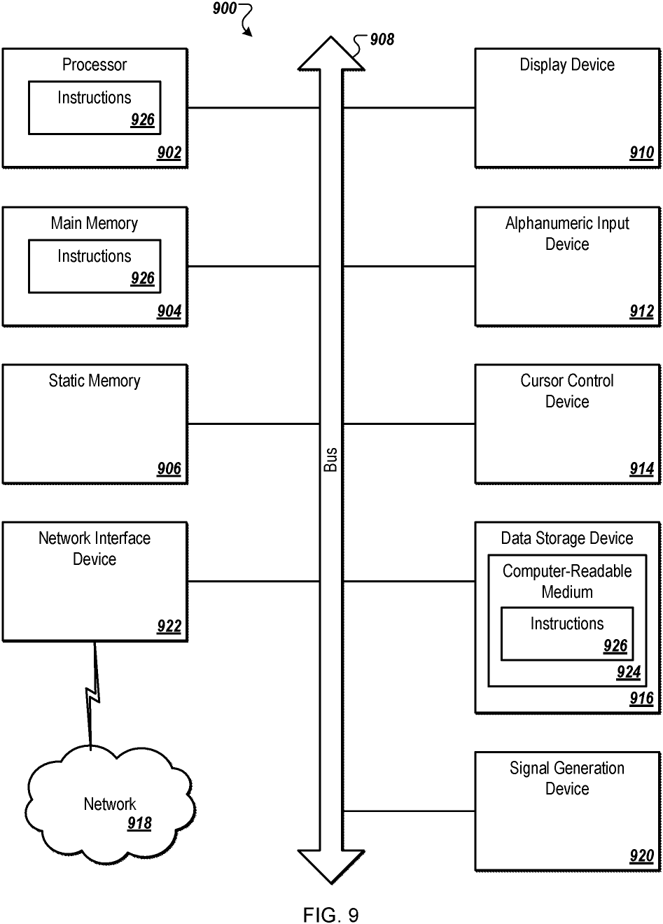

The method 600 may be performed by processing logic that may include hardware (circuitry, dedicated logic, etc.), software (such as is run on a computer system or a dedicated machine), or a combination of both, which processing logic may be included in the processing device 902 of FIG. 9, the communication system 500 of FIG. 5, or another device, combination of devices, or systems.

The method 600 may begin at block 605 where the processing logic may be configured to receive an incoming notification and request an input in response to the incoming notification. At block 610, the processing logic may be configured to receive input comprising EEG data. At block 615, the processing logic may be configured to determine a command based on the input. At block 620, the processing logic may be configured to cause an action to be performed based on the command.

Modifications, additions, or omissions may be made to the method 600 without departing from the scope of the present disclosure. For example, in some embodiments, the method 600 may include any number of other components that may not be explicitly illustrated or described.

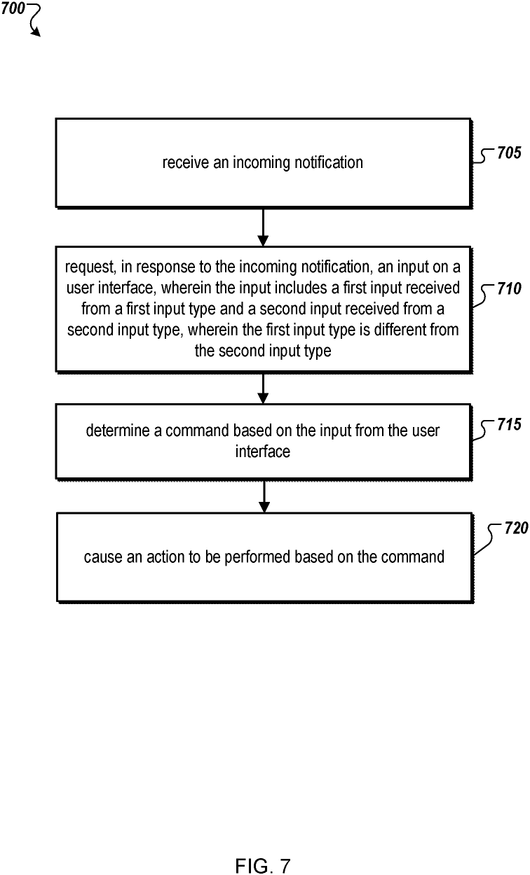

FIG. 7 illustrates a process flow of an example method 700 that may be used by an agent, in accordance with at least one embodiment described in the present disclosure. The method 700 may be arranged in accordance with at least one embodiment described in the present disclosure.

The method 700 may be performed by processing logic that may include hardware (circuitry, dedicated logic, etc.), software (such as is run on a computer system or a dedicated machine), or a combination of both, which processing logic may be included in the processing device 902 of FIG. 9, the communication system 500 of FIG. 5, or another device, combination of devices, or systems.

The method 700 may begin at block 705 where the processing logic may cause an agent to receive an incoming notification.

At block 710, the processing logic may cause an agent to request, in response to the incoming notification, an input on a user interface, wherein the input includes a first input received from a first input type and a second input received from a second input type, wherein the first input type is different from the second input type.

At block 715, the processing logic may cause an agent to determine a command based on the input from the user interface

At block 720, the processing logic may cause an agent to cause an action to be performed based on the command.

Modifications, additions, or omissions may be made to the method 700 without departing from the scope of the present disclosure. For example, in some embodiments, the method 700 may include any number of other components that may not be explicitly illustrated or described.

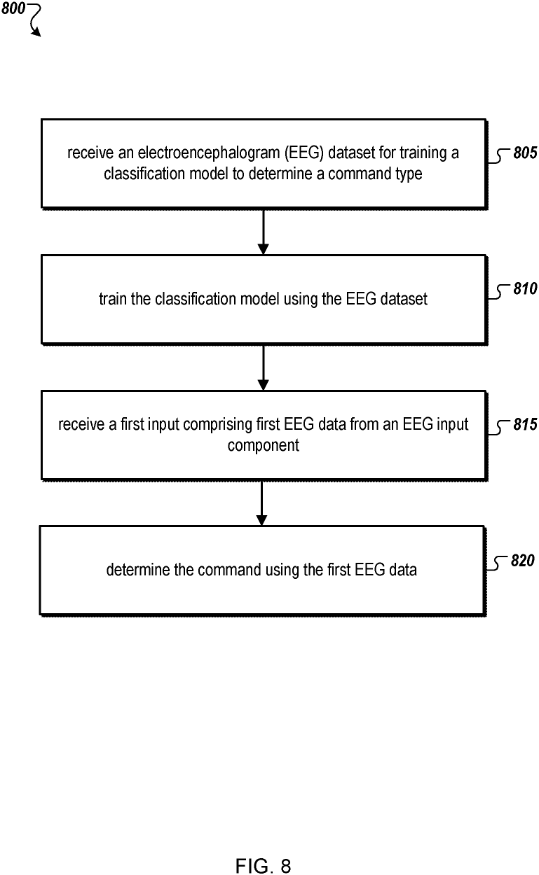

FIG. 8 illustrates a process flow of an example method 800 that may be used for a virtual assist device, in accordance with at least one embodiment described in the present disclosure. The method 800 may be arranged in accordance with at least one embodiment described in the present disclosure.

The method 800 may be performed by processing logic that may include hardware (circuitry, dedicated logic, etc.), software (such as is run on a computer system or a dedicated machine), or a combination of both, which processing logic may be included in the processing device 902 of FIG. 9, the communication system 500 of FIG. 5, or another device, combination of devices, or systems.

The method 800 may begin at block 805 where the processing logic may be configured to receive an electroencephalogram (EEG) dataset for training a classification model to determine a command type.

At block 810, the processing logic may be configured to train the classification model using the EEG dataset.

At block 815, the processing logic may be configured to receive a first input comprising first EEG data from an EEG input component.

At block 820, the processing logic may be configured to determine the command using the first EEG data.

Modifications, additions, or omissions may be made to the method 800 without departing from the scope of the present disclosure. For example, in some embodiments, the method 800 may include any number of other components that may not be explicitly illustrated or described.

For simplicity of explanation, methods and/or process flows described herein are depicted and described as a series of acts. However, acts in accordance with this disclosure may occur in various orders and/or concurrently, and with other acts not presented and described herein. Further, not all illustrated acts may be used to implement the methods in accordance with the disclosed subject matter. In addition, those skilled in the art will understand and appreciate that the methods may alternatively be represented as a series of interrelated states via a state diagram or events. Additionally, the methods disclosed in this specification are capable of being stored on an article of manufacture, such as a non-transitory computer-readable medium, to facilitate transporting and transferring such methods to computing devices. The term article of manufacture, as used herein, is intended to encompass a computer program accessible from any computer-readable device or storage media. Although illustrated as discrete blocks, various blocks may be divided into additional blocks, combined into fewer blocks, or eliminated, depending on the desired implementation.

FIG. 9 illustrates a diagrammatic representation of a machine in the example form of a computing device 900 within which a set of instructions, for causing the machine to perform any one or more of the methods discussed herein, may be executed. The computing device 900 may include a rackmount server, a router computer, a server computer, a mainframe computer, a laptop computer, a tablet computer, a desktop computer, or any computing device with at least one processor, etc., within which a set of instructions, for causing the machine to perform any one or more of the methods discussed herein, may be executed. In alternative embodiments, the machine may be connected (e.g., networked) to other machines in a LAN, an intranet, an extranet, or the Internet. The machine may operate in the capacity of a server machine in client-server network environment. Further, while only a single machine is illustrated, the term “machine” may also include any collection of machines that individually or jointly execute a set (or multiple sets) of instructions to perform any one or more of the methods discussed herein.

The example computing device 900 includes a processing device (e.g., a processor) 902, a main memory 904 (e.g., read-only memory (ROM), flash memory, dynamic random access memory (DRAM) such as synchronous DRAM (SDRAM)), a static memory 906 (e.g., flash memory, static random access memory (SRAM)) and a data storage device 916, which communicate with each other via a bus 908.

Processing device 902 represents one or more general-purpose processing devices such as a microprocessor, central processing unit, or the like. More particularly, the processing device 902 may include a complex instruction set computing (CISC) microprocessor, reduced instruction set computing (RISC) microprocessor, very long instruction word (VLIW) microprocessor, or a processor implementing other instruction sets or processors implementing a combination of instruction sets. The processing device 902 may also include one or more special-purpose processing devices such as an application specific integrated circuit (ASIC), a field programmable gate array (FPGA), a digital signal processor (DSP), network processor, or the like. The processing device 902 is configured to execute instructions 926 for performing the operations and steps discussed herein.

The computing device 900 may further include a network interface device 922 which may communicate with a network 918. The computing device 900 also may include a display device 910 (e.g., a liquid crystal display (LCD) or a cathode ray tube (CRT)), an alphanumeric input device 912 (e.g., a keyboard), a cursor control device 914 (e.g., a mouse) and a signal generation device 920 (e.g., a speaker). In at least one embodiment, the display device 910, the alphanumeric input device 912, and the cursor control device 914 may be combined into a single component or device (e.g., an LCD touch screen).

The data storage device 916 may include a computer-readable storage medium 924 on which is stored one or more sets of instructions 926 embodying any one or more of the methods or functions described herein. The instructions 926 may also reside, completely or at least partially, within the main memory 904 and/or within the processing device 902 during execution thereof by the computing device 900, the main memory 904 and the processing device 902 also constituting computer-readable media. The instructions may further be transmitted or received over a network 918 via the network interface device 922.

While the computer-readable storage medium 924 is shown in an example embodiment to be a single medium, the term “computer-readable storage medium” may include a single medium or multiple media (e.g., a centralized or distributed database and/or associated caches and servers) that store the one or more sets of instructions. The term “computer-readable storage medium” may also include any medium that is capable of storing, encoding or carrying a set of instructions for execution by the machine and that cause the machine to perform any one or more of the methods of the present disclosure. The term “computer-readable storage medium” may accordingly be taken to include, but not be limited to, solid-state memories, optical media and magnetic media.

In some embodiments, the different components, modules, engines, and services described herein may be implemented as objects or processes that execute on a computing system (e.g., as separate threads). While some of the systems and methods described herein are generally described as being implemented in software (stored on and/or executed by hardware), specific hardware implementations or a combination of software and specific hardware implementations are also possible and contemplated.

Terms used herein and especially in the appended claims (e.g., bodies of the appended claims) are generally intended as “open” terms (e.g., the term “including” should be interpreted as “including, but not limited to,” the term “having” should be interpreted as “having at least,” the term “includes” should be interpreted as “includes, but is not limited to,” etc.).

Additionally, if a specific number of an introduced claim recitation is intended, such an intent will be explicitly recited in the claim, and in the absence of such recitation no such intent is present. For example, as an aid to understanding, the following appended claims may contain usage of the introductory phrases “at least one” and “one or more” to introduce claim recitations. However, the use of such phrases should not be construed to imply that the introduction of a claim recitation by the indefinite articles “a” or “an” limits any particular claim containing such introduced claim recitation to embodiments containing only one such recitation, even when the same claim includes the introductory phrases “one or more” or “at least one” and indefinite articles such as “a” or “an” (e.g., “a” and/or “an” should be interpreted to mean “at least one” or “one or more”); the same holds true for the use of definite articles used to introduce claim recitations.

In addition, even if a specific number of an introduced claim recitation is explicitly recited, it is understood that such recitation should be interpreted to mean at least the recited number (e.g., the bare recitation of “two recitations,” without other modifiers, means at least two recitations, or two or more recitations). Furthermore, in those instances where a convention analogous to “at least one of A, B, and C, etc.” or “one or more of A, B, and C, etc.” is used, in general such a construction is intended to include A alone, B alone, C alone, A and B together, A and C together, B and C together, or A, B, and C together, etc. For example, the use of the term “and/or” is intended to be construed in this manner.

Further, any disjunctive word or phrase presenting two or more alternative terms, whether in the description, claims, or drawings, should be understood to contemplate the possibilities of including one of the terms, either of the terms, or both terms. For example, the phrase “A or B” should be understood to include the possibilities of “A” or “B” or “A and B.”

Additionally, the use of the terms “first,” “second,” “third,” etc., are not necessarily used herein to connote a specific order or number of elements. Generally, the terms “first,” “second,” “third,” etc., are used to distinguish between different elements as generic identifiers. Absence a showing that the terms “first,” “second,” “third,” etc., connote a specific order, these terms should not be understood to connote a specific order. Furthermore, absence a showing that the terms first,” “second,” “third,” etc., connote a specific number of elements, these terms should not be understood to connote a specific number of elements. For example, a first widget may be described as having a first side and a second widget may be described as having a second side. The use of the term “second side” with respect to the second widget may be to distinguish such side of the second widget from the “first side” of the first widget and not to connote that the second widget has two sides.

All examples and conditional language recited herein are intended for pedagogical objects to aid the reader in understanding the invention and the concepts contributed by the inventor to furthering the art, and are to be construed as being without limitation to such specifically recited examples and conditions. Although embodiments of the present disclosure have been described in detail, it should be understood that the various changes, substitutions, and alterations could be made hereto without departing from the spirit and scope of the present disclosure.

What is claimed is:

1. A virtual assist device, comprising:
an agent configured to receive an incoming notification and request an input in response to the incoming notification;
an electroencephalogram (EEG) input component configured to receive the input comprising EEG data and send the input to the agent,
wherein the agent is configured to determine a command based on the input; and
a communication unit configured to cause an action to be performed based on the command.

2. The virtual assist device of claim 1, wherein the agent is further configured to:
determine the command by:
training a classification model to determine the command using
training input comprising an EEG dataset, and
identifying the command using the classification model.

3. The virtual assist device of claim 2, wherein the classification model is one or more of: a convolutional neural network, a long term short memory network, a recurrent neural network, a sequence to sequence model, or a transformer model.

4. The virtual assist device of claim 1, wherein the EEG input component is further configured to:
determine the command is one or more of: an affirmative command, a negative command, a word, a numbered option, a directional option, an assistance-activating command, or a password for authenticating a user.

5. The virtual assist device of claim 1, wherein the agent is further configured to:
identify a notification type for the incoming notification;
determine a notification time for the incoming notification based on the notification type; and
request the input on the user interface based on the notification time.

6. The virtual assist device of claim 1, further comprising a wearable device housing the EEG input component, the communication unit, and the agent, wherein the EEG input component comprises one or more sensors configured to contact a user at a selected cranial position to receive the EEG data used to determine the command.

7. The virtual assist device of claim 1, wherein the input is requested using one or more of: a sound, haptic feedback, an electrical stimulation, a magnetic stimulation, or a visual stimulation.

8. The virtual assist system of claim 1, further comprising an additional input type comprising one or more of: electromyography (EMG) input, magnetoencephalography (MEG) input, electrocardiogram (ECG) input, or photoplethysmography (PPG) input, microphone input, vibration sensor input, accelerometer input, a capacitive input, a resistive input, or a button click, wherein the additional input type is used to determine the command.

9. A computer-readable storage medium including computer executable instructions that, when executed by one or more processors, cause an agent to:
receive an incoming notification;
request, in response to the incoming notification, an input on a user interface, wherein the input includes a first input received from a first input type and a second input received from a second input type, wherein the first input type is different from the second input type;
determine a command based on the input from the user interface; and
cause an action to be performed based on the command.

10. The computer-readable storage medium of claim 9, further comprising instructions that, when executed by one or more processors, cause the agent to:
identify a notification type for the incoming notification;
determine a notification time for the incoming notification based on the notification type;
request the input on the user interface based on the notification time.

11. The computer-readable storage medium of claim 9, further comprising instructions that, when executed by one or more processors, cause the agent to:
determine the command in less than a threshold amount of time after receiving the input from the user interface, wherein the threshold amount of time is less than one or more of: 1 s, 1 ms, 100 µs, 10 µs, or 1 µs.

12. The computer-readable storage medium of claim 9, further comprising instructions that, when executed by one or more processors, cause the agent to:
determine the command by:
training a classification model to determine the command using training input comprising an EEG dataset, and
identifying the command using the classification model.

13. The computer-readable storage medium of claim 9, further comprising instructions that, when executed by one or more processors, cause the agent to:
determine the command is one or more of: an affirmative command, a negative command, a word, a numbered option, a directional option, an assistance-activating command, or a password for authenticating a user.

14. The computer-readable storage medium of claim 9, wherein first input type is an electroencephalogram (EEG) input, and the second input type is one or more of:
electromyography (EMG) input, magnetoencephalography (MEG) input, electrocardiogram (ECG) input, or photoplethysmography (PPG) input, microphone input, vibration sensor input, accelerometer input, a capacitive input, a resistive input, or a button click, wherein the additional input type is used to determine the command.

15. A computer-implemented method, comprising:
receiving an electroencephalogram (EEG) dataset for training a classification model to determine a command type;
training the classification model using the EEG dataset;
receiving a first input comprising first EEG data from an EEG input component;
determining the command using the first EEG data.

16. The computer-implemented method of claim 15, further comprising:
receiving a second input dataset for refining the classification model to determine the command type, wherein the second input dataset is not EEG data; and refining the classification model using the second input dataset.

17. The computer-implemented method of claim 16, further comprising:
receiving second input data from a second input component, wherein the second input data is not EEG data; and
determining the command using the second input data.

18. The computer-implemented method of claim 15, wherein the classification model is one or more of: a convolutional neural network, a long term short memory network, a recurrent neural network, a sequence to sequence model, or a transformer model.

19. The computer-implemented method of claim 15, further comprising:
determining the command is one or more of: an affirmative command, a negative command, a word, a numbered option, a directional option, an assistance-activating command, or a password for authenticating a user.

20. The computer-implemented method of claim 15, further comprising:
monitoring a second input channel for a second input component and a third input channel for a third input component;
identifying second input data from the second input channel and third input data from the third input component; and
determining the command using one or more of the second input data or the third input data.
